(19) United States
(12) Patent Application Publication
(10) Pub. No.: US 2023/0357103 A1
(43) Pub. Date: Nov. 9, 2023

(54) CYCLOHEXYLBENZENE PRODUCTION METHOD AND CYCLOHEXYLBENZENE COMPOSITION USING SAME

(71) Applicant: ENEOS Corporation, Tokyo (JP)

(72) Inventors: Yasuyuki IWASA, Tokyo (JP); Yasuhiro ARAKI, Tokyo (JP)

(73) Assignee: ENEOS Corporation, Tokyo (JP)

(21) Appl. No.: 18/252,439

(22) PCT Filed: Nov. 9, 2021
(86) PCT No.: PCT/JP2021/041111

§ 371 Date: May 10, 2023

(30) Foreign Application Priority Data
Nov. 13, 2020 (JP) 2020-189699

(51) Int. Cl. C07C 2/72

(52) U.S. Cl. CPC C07C 2/72; C07C 2529/06

(57) ABSTRACT

To provide a method for producing cyclohexylbenzene, which is capable of obtaining cyclohexylbenzene at a high selectivity, and a cyclohexylbenzene composition obtained with the method. A method for producing cyclohexylbenzene, comprising a step of bringing a raw material containing benzene and cyclohexene or cyclohexanol into contact with a solid acid catalyst to thereby perform alkylation reaction, wherein the solid acid catalyst is a silica-alumina catalyst or an MTW-type zeolite catalyst.

### BACKGROUND OF THE INVENTION

### Field of the Invention

The present invention relates to a method for producing cyclohexylbenzene and a cyclohexylbenzene composition obtained with the method.

### Background Art

Cyclohexylbenzenes are widely used in overcharge inhibitors for lithium ion batteries, and are industrially useful susbtances. Examples of known methods for producing cyclohexylbenzenes include methods for producing cyclohexylbenzenes by hydrogenation/dimerization of benzenes and methods for producing cyclohexylbenzenes by partial hydrogenation of biphenyls, and methods for efficiently producing cyclohexylbenzenes are variously studied.

Patent Literature 1 discloses a method for producing cyclohexylbenzene by reacting benzene with cyclohexene and thus generating cyclohexylbenzene and at least one poly cyclohexylbenzene.

Patent Literature 2 discloses a method for producing cyclohexylbenzene by contacting benzene and a cyclic monoolefin with an alkylation catalyst.

### CITATION LIST

### Patent Literature

[Patent Literature 1] US 8921604 B
[Patent Literature 1] US 9233887 B

### SUMMARY OF THE INVENTION

### Technical Problem

However, the method of Patent Literature 1 is a method which causes generation of a large amount of by-products, and requires another step of producing additional cyclohexylbenzene by transalkylation of polycyclohexylbenzene as a by-product. Thus, complicated steps and additional production facilities are needed, and there is room for improvement from the viewpoint of industrial production of cyclohexylbenzene at a good efficiency.

Furthermore, the conventional methods for producing cyclohexylbenzene as in Patent Literature 1 and Patent Literature 2 are not sufficient in terms of the conversion of a raw material and the selectivity and purity of cyclohexylbenzene, and have room for improvement.

An object of the present invention is to provide a method for producing cyclohexylbenzene, which is capable of obtaining cyclohexylbenzene at a high selectivity, and a cyclohexylbenzene composition obtained with the method.

### Solution to Problem

The present inventors have made intensive studies in order to solve the above problems, and as a result, have found that the above problems are solved by bringing benzene and cyclohexene or cyclohexanol into contact with a specified solid acid catalyst, and thus have completed the present invention.

Specifically, the present invention provides the following various specific aspects.

[1] A method for producing cyclohexylbenzene, comprising a step of bringing a raw material containing benzene and cyclohexene or cyclohexanol into contact with a solid acid catalyst to thereby perform alkylation reaction, wherein

the solid acid catalyst is a silica-alumina catalyst or an MTW-type zeolite catalyst.

The method for producing cyclohexylbenzene according to [1], wherein the solid acid catalyst is a silica-alumina catalyst, and the raw material contains the cyclohexanol.

The method for producing cyclohexylbenzene according to [1], wherein the solid acid catalyst is an MTW-type zeolite, and the raw material contains the cyclohexene.

The method for producing cyclohexylbenzene according to any one of [1] to [3], wherein the alkylation reaction is performed at a pressure of 0.5 to 3.0 MPaG, a temperature of 150 to 400° C., and a weight hourly space velocity (WHSV) of 1.0 h⁻¹ to 100 h⁻¹.

The method for producing cyclohexylbenzene according to any one of [1] to [4], wherein the raw material contains 70% by mass or more of the benzene.

A cyclohexylbenzene composition obtained by the method for producing cyclohexylbenzene according to any one of [1] to [5], wherein the content of cyclohexylbenzene is 97.0% by mass or more and the content of bicyclohexyl is 1.0% by mass or less.

### Advantageous Effects of Invention

The present invention can provide a method for producing cyclohexylbenzene, which is capable of obtaining cyclohexylbenzene at a high selectivity, and a cyclohexylbenzene composition obtained with the method.

### DETAILED DESCRIPTION OF THE INVENTION

Hereinafter, a production method and a cyclohexylbenzene composition obtained with the method, according to embodiments of the present invention, are described. The scope of the present invention is not limited to the following embodiments, and can be arbitrarily modified within the technical idea of the present invention. Herein, a numerical value range expressed with “to” means a range including numerical values described before and after “to” as the lower limit value and the upper limit value.

The method for producing cyclohexylbenzene according to the present embodiment includes a step of bringing a raw material containing benzene and cyclohexene or cyclohexanol into contact with a solid acid catalyst to thereby perform alkylation reaction.

### (Raw Material)

In the production method according to the present embodiment, benzene and cyclohexene or cyclohexanol are used in the raw material. The benzene, cyclohexene and cyclohexanol may be produced by ordinary methods and then used, or commercially available products may also be obtained and then used. The benzene, cyclohexene and cyclohexanol are not particularly limited, and preferably each have a purity of about more than 99%, more preferably about more than 99.9% on a mass basis from the viewpoint of suppression of generation of any by-product.

### (Solid Acid Catalyst)

The solid acid catalyst may be any catalyst as long as it has activity for the alkylation reaction of the benzene. Examples of the solid acid catalyst include inorganic solid acid catalysts such as: activated white earth; silica/alumina; silicamagnesium; zeolite; zirconia; silica; titania; ceria; and sodium diphosphate; and organic solid acid catalysts such as a strong acidic ion exchange resin. Among them, the solid acid catalyst here used is preferably an inorganic solid acid catalyst, and a silica-alumina catalyst and a zeolite catalyst are more preferable because cyclohexylbenzene tends to be able to be produced at a high efficiency. The zeolite catalyst here used is particularly preferably an MTW-type zeolite catalyst. The solid acid catalyst may be used singly or in combination of a plurality thereof.

The MTW-type zeolite catalyst means a zeolite catalyst corresponding to MTW as a structure code in the database of the International Zeolite Association. The MTW-type zeolite catalyst is known to have a 12-membered oxygen ring structure and have an average pore size of about 6.5 Å or less. The 12-membered oxygen ring structure tends to allow the raw material to easily enter pores and allow reaction to efficiently progress. The average pore size is about 6.5 Å or less to result in tendencies to enable condensation of heavy matter in the system and thus progress of coking to be suppressed and enable deterioration in activity due to catalyst degradation to be suppressed. Thus, the solid acid catalyst here used is particularly preferably the MTW-type zeolite catalyst among zeolite catalysts. Whether or not the MTW-type zeolite structure is contained can be confirmed by, for example, X-ray diffraction.

Hereinafter, an embodiment is described in which the silica-alumina catalyst and/or the MTW-type zeolite catalyst are/is used in the solid acid catalyst.

### Silica-Alumina Catalyst and/or MTW-type Zeolite Catalyst

The silica-alumina catalyst and/or the MTW-type zeolite catalyst here used may be commercially available product(s), or may also be synthesized by a known method.

Specific examples of the MTW-type zeolite catalyst include ZSM-12, CZH-5, NU-13, TPZ-12, Theta-3 and VS-12, and in particular, ZSM-12 is more preferable from the viewpoint of reactivity of the alkylation reaction.

In the production method according to the present embodiment, the MTW-type zeolite catalyst here used can be particularly suitably, for example, small crystal/highly active ZSM-12 increased in activity due to conversing of aromatic hydrocarbon, as disclosed in JP 2006-506311 A.

The silica/alumina ratio in the silica-alumina catalyst and/or the MTW-type zeolite catalyst is not particularly limited because an appropriate value thereof is varied depending on the type of the catalyst(s) and reaction conditions. The silica/alumina ratio in the silica-alumina catalyst is not particularly limited, and is preferably 0.5 to 10, more preferably 1 to 5. The silica/alumina ratio in the MTW-type zeolite catalyst is not particularly limited, and is preferably 30 to 200, more preferably 30 to 100. When the silica/alumina ratio is in the range, catalyst activity tends to be excellent. When the silica/alumina ratio is in the range, water resistance of the silica-alumina catalyst and/or the MTW-type zeolite catalyst tends to be sufficient in the case of the presence of water as a by-product in the reaction system.

The molar ratio of silica/alumina can be measured by, for example, an atomic absorption method, a fluorescence X-ray diffraction method, or ICP (induction coupled plasma) emission spectroscopy.

A metal atom as an auxiliary component may be supported on the silica-alumina catalyst and/or the MTW-type zeolite catalyst. Examples of the metal atom as an auxiliary component include a metal having the effect of enhancing catalyst activity and a metal having the effect of suppressing carbon deposition.

Examples of the metal atom as an auxiliary component include magnesium, calcium, strontium, barium, lanthanum, cerium, praseodymium, neodymium, samarium, europium, gadolinium, terbium, dysprosium, holmium, erbium, thulium, ytterbium, lutetium, titanium, vanadium, chromium, molybdenum, tungsten, manganese, rhenium, iron, ruthenium, cobalt, rhodium, iridium, nickel, palladium, platinum, copper, silver, zinc, aluminum, indium, germanium, tin, lead, phosphorus, antimony, bismuth and selenium. The metal atom as an auxiliary component may be used singly or in combination of a plurality thereof. The amount of the metal atom supported is not particularly limited, and is usually 0.1 to 20% by mass based on the total amount of the silica-alumina catalyst and/or the MTW-type zeolite catalyst. The method for supporting the metal atom is not particularly limited, and can be conducted by a known method. Examples of the supporting method include an ion exchange method, an impregnation method, and a mixing method. The metal source used in the supporting of the metal atom can be, for example, nitrate, sulfate, chloride, acetate, or formate.

The silica-alumina catalyst and/or the MTW-type zeolite catalyst may further contain a formation aid from the viewpoint of an enhancement in formability as long as the object of the present invention is not impaired. The formation aid may be, for example, at least one selected from the group consisting of a thickener, a surfactant, a water retention agent, a plasticizer, a binder raw material, and the like. The silica-alumina catalyst and/or the MTW-type zeolite catalyst may contain other useful component as long as the object of the present invention is not impaired.

The silica-alumina catalyst and/or the MTW-type zeolite catalyst are/is preferably subjected to dehydration treatment in advance and then used because moisture may adsorb to the catalyst(s). The dehydration treatment is performed by, for example, heating at a temperature of 100 to 300° C. for several hours.

### Method for Producing Cyclohexylbenzene

The production method according to the present embodiment is represented by the following reaction schemes (1) and (2).

The production method by the following reaction scheme (1) according to the present embodiment is a method for producing cyclohexylbenzene by bringing benzene and cyclohexene with the silica-alumina catalyst and/or the MTW-type zeolite catalyst to thereby perform alkylation reaction.

[Chem. 1]

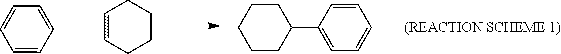

The MTW-type zeolite catalyst is particularly preferably used in the production method by the reaction scheme (1) according to the present embodiment, without particular limitation, because cyclohexylbenzene tends to be able to be obtained at a higher selectivity.

The production method by the following reaction scheme (2) according to the present embodiment is a method for producing cyclohexylbenzene by bringing benzene and cyclohexanol with the silica-alumina catalyst and/or the MTW-type zeolite catalyst to thereby perform alkylation reaction.

[Chem. 2]

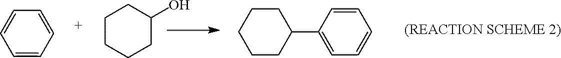

The silica-alumina catalyst is particularly preferably used in the production method by the reaction scheme (2) according to the present embodiment, without particular limitation. The production method by the reaction scheme (2) generates water as a by-product, and thus use of the silica-alumina catalyst which tends to be high in water resistance tends to provide cyclohexylbenzene at a higher selectivity over a long period of time.

The production method according to the present embodiment, described above, preferably produces cyclohexylbenzene by continuous feeding of the raw material to the reaction system in which the solid acid catalyst is present.

The reaction system is not particularly limited as long as it has a structure which has a moiety for retention of the catalyst packed, which can continuously feed the raw material, and which discharges cyclohexylbenzene (and, if necessary, by-product) produced, outside the system. Examples of the reaction system include a tube reactor, a tank reactor, a fixed bed-type reactor, and a fluid bed-type reactor, and in particular, a tube reactor, a fixed bed reactor, and a tank reactor are preferable.

One example of the production method according to the present embodiment, including the reaction system, is shown below.

The raw material is continuously fed to the reaction system in which the silica-alumina catalyst and/or the MTW-type zeolite catalyst are/is present, and the alkylation reaction of benzene is performed. A reaction product after the alkylation reaction is discharged outside the reaction system, and cyclohexylbenzene is separated and purified from the reaction product discharged.

Herein, the unreacted raw material may be recovered from the reaction product after separation of cyclohexylbenzene, and the raw material recovered may be mixed with a new raw material and the mixture may be recycled.

The content of benzene in the raw material in the production method according to the present embodiment is not particularly limited, and is preferably 50% by mass or more, more preferably 70% by mass or more, further preferably 90% by mass or more, particularly preferably 95% by mass or more.

The upper limit of the content of benzene is not particularly limited, and is usually 99% by mass or less from the economic viewpoint.

In other words, the mass ratio between benzene and cyclohexene and/or cyclohexanol in the raw material is preferably 50:50 to 99:1, preferably 70:30 to 99:1, more preferably 90:10 to 99:1, further preferably 95:5 to 99:1.

The above range tends to enable generation of any by-product to be suppressed and enable cyclohexylbenzene to be produced at a high selectivity.

The raw material fed to the reaction system is preferably a gas. The raw material, when is a gas, can be mixed with a gas other than the raw material and fed as a raw material-containing gas to the reaction system. The gas other than the raw material may be any gas as long as it does not inhibit the alkylation reaction, and examples thereof include a nitrogen gas, an argon gas, a neon gas, and a carbon dioxide gas. The gas other than the raw material, to be fed to the reaction system, preferably includes substantially no molecular hydrogen. The “including substantially no molecular hydrogen” means no intended feeding of any molecular hydrogen to the reaction system. More specifically, it is meant that the content of molecular hydrogen is preferably 0 to 3% by mass, more preferably 0 to 1% by mass, further preferably 0 to 0.5% by mass based on the total mass of the raw material fed to the reaction system.

A diluent substantially inactive, in addition to the gas other than the raw material, may be fed in the alkylation reaction under alkylation conditions. Examples of the diluent include linear paraffin-based hydrocarbon, branched paraffin-based hydrocarbon, and cyclic paraffin-based hydrocarbon.

The weight hourly space velocity (WHSV) of the raw material-containing gas is not particularly limited, and is preferably 1.0 h⁻¹ or more, more preferably 3.0 h⁻¹ or more, further preferably 5.0 h⁻¹ or more. The weight hourly space velocity of the raw material-containing gas is preferably 100 h⁻¹ or less, more preferably 50 h⁻¹ or less, further preferably 20 h⁻¹ or less. When the weight hourly space velocity is in the range, cyclohexylbenzene tends to be able to be obtained at a high selectivity over a long period of time. The WHSV is a ratio (F/W) of the feeding velocity (feeding weight/time) F to the raw material to the weight W of the solid acid catalyst in the reaction system.

The reaction temperature of the alkylation reaction is not particularly limited, and is preferably 150° C. or more, more preferably 200° C. or more, further preferably 250° C. or more. The reaction temperature of the alkylation reaction is preferably 400° C. or less, more preferably 380° C. or less, further preferably 350° C. or less.

When the reaction temperature is the lower limit or more, the reaction velocity of the alkylation reaction tends to be sufficient to result in an increase in conversion of cyclohexene or hexanol. When the reaction temperature is the upper limit or less, generation of a by-product tends to be suppressed to thereby enable cyclohexylbenzene to be obtained at a high selectivity.

The reaction pressure of the alkylation reaction is not particularly limited, and is preferably atmospheric pressure or more, more preferably 0.3 MPaG or more, further preferably 0.5 MPaG or more. The reaction pressure of the alkylation reaction is preferably 10 MPaG or less, more preferably 5 MPaG or less, further preferably 3 MPaG or less.

The method for separating and purifying cyclohexylbenzene from the reaction product is not particularly limited, and can be purified by a known distillation operation or the like.

The reaction product obtained by the alkylation reaction tends to include, besides the raw material and cyclohexylbenzene, dicyclohexylbenzene, a dimer other than dicyclohexylbenzene, bicyclohexyl, and/or the like. It is known that bicyclohexyl has a boiling point close to that of cyclohexylbenzene and thus is difficult to separate by a distillation operation. The production method according to the present embodiment can produce cyclohexylbenzene at a high selectivity, and thus the amount of bicyclohexyl generated is very small. Thus, the production method of the present embodiment is an industrially excellent method, and can efficiently produce cyclohexylbenzene.

As described above, the production method according to the present embodiment can provide cyclohexylbenzene at a high conversion of cyclohexene or hexanol at a high selectivity with suppression of side reaction.

### (Cyclohexylbenzene Composition)

The cyclohexylbenzene composition according to the present embodiment is a composition including cyclohexylbenzene produced with the above production method. The cyclohexylbenzene composition according to the present embodiment preferably has a boiling point ranging from 200° C. to 250° C., more preferably a boiling point ranging from 230° C. to 250° C. The content of cyclohexylbenzene in the cyclohexylbenzene composition is preferably 95% by mass or more, more preferably 97% by mass or more, further preferably 99% by mass or more. On the other hand, bicyclohexyl has a boiling point close to that of cyclohexylbenzene and is difficult to separate by a distillation operation, and thus a lower content of bicyclohexyl in the cyclohexylbenzene composition is more preferable. Thus, the content of bicyclohexyl in the cyclohexylbenzene composition is preferably 2.0% mass or less, more preferably 1.0% by mass or less, further preferably 0.5% by mass.

### EXAMPLES

Hereinafter, the present invention is more specifically described with reference to Examples, but the present invention is not limited to these Examples.

### Example 1

### (Preparation of Proton-Type ZSM-12 as MTW-Type Zeolite Catalyst)

An aqueous tetraethylammonium hydroxide (TEAOH) solution (concentration 20%), sodium aluminate, colloidal silica (Snowtex NS, manufactured by Nissan Chemical Corporation, silica concentration 20%) and ion-exchanged water were mixed so that Na₂O: Al₂O₃: SiO₂: TEAOH: H₂O = 1:1:60:13:800 was satisfied, and the mixture was stirred until a uniform system was achieved. The mixture was placed in an autoclave having a cylinder made of Teflon, and subjected to hydrothermal synthesis at 160° C. for 120 hours. The resulting product was washed and subjected to filtration, and then fired at 550° C. for 4 hours, to obtain Na-type ZSM-12 zeolite. The Na-type ZSM-12 obtained was stirred in 20-fold by mass of an aqueous 1 mol/L ammonium nitrate at 95° C. for 2 hours and subjected to ion exchange treatment. After the ion exchange treatment was performed twice, the resultant was washed and subjected to filtration, and fired at 550° C. for 4 hours, to obtain proton-type ZSM-12 zeolite.

### (Production of Cyclohexylbenzene)

A tube reactor was filled with 1.0 g of the proton-type ZSM-12, and a reaction tube was connected to a fixed bed fluid-type reaction apparatus. The reaction tube was heated to 250° C. under flow of nitrogen at 23 mL/min, and then retained for 1 hour. Thereafter, benzene and cyclohexene (benzene: cyclohexene = 95:5 (mass ratio)) in the raw material, and nitrogen were each fed to the reactor, and the alkylation reaction of benzene was performed at a reaction temperature of 250° C. and a reaction pressure of 0.9 MPaG. The compositional ratio in feeding to the reactor was as follows: raw material: nitrogen = 1:1 (molar ratio). The WHSV was 5.0 h⁻¹.

The reaction product of the alkylation reaction was taken from the tube reactor after a lapse of 4 hours from the point of time of the start of the reaction. The start of the reaction was here the point of time at which feeding of the raw material was started. The reaction product taken was analyzed with gas chromatograph (FID-GC) provided with a hydrogen flame detector. The amount of each component in the reaction product taken was quantitatively determined based on the gas chromatograph, and the conversion of cyclohexene and the selectivity of each of components including cyclohexylbenzene were calculated. Furthermore, the reaction product taken was subjected to distillation under reduced pressure at 100 mmHg and 160° C. to 165° C., to obtain a cyclohexylbenzene composition. The content of cyclohexylbenzene and the content of bicyclohexyl in the cyclohexylbenzene composition were calculated. The results are shown in Table 1.

The conversion of cyclohexene was calculated according to the following formula (1).

Conversion of cyclohexene = (Mass of cyclohexene included in reaction product/Mass of cyclohexene included in raw material) × 100 ... (1)

The selectivity of cyclohexylbenzene was calculated according to the following formula (2).

Selectivity of cyclohexylbenzene = (Mass of cyclohexylbenzene included in reaction product/Total mass of all components included in reaction product) × 100 ... (2)

The selectivity of each of dicyclohexylbenzene, a dimer other than cyclohexylbenzene, and bicyclohexyl was calculated by the same method as in the selectivity of cyclohexylbenzene.

### Examples 2 to 4

The same manner as in Example 1 was performed except that the mass ratio between benzene and cyclohexene in the raw material was changed to each value described in Table 1. The results are shown in Table 1.

### Example 5

The same manner as in Example 1 was performed except that cyclohexanol was used instead of cyclohexene. The results are shown in Table 1. Herein, the conversion of cyclohexanol was calculated by the same method as in the conversion of cyclohexene.

### Example 6

The same manner as in Example 1 was performed except that silica/alumina (model number: N632NH, manufactured by JGC C&C) was used instead of ZSM-12. The results are shown in Table 2.

### Example 7

The same manner as in Example 6 was performed except that cyclohexanol was used instead of cyclohexene. The results are shown in Table 2.

### Examples 8 and 9

The same manner as in Example 1 was performed except that the reaction temperature and the reaction pressure were changed to respective values described in Table 1. The results are shown in Table 2.

### Comparative Example 1

The same manner as in Example 1 was performed except that ZSM-5 (model number: HSZ842HODC, manufactured by Tosoh Corporation) as the MFI-type zeolite catalyst was used instead of ZSM-12. The results are shown in Table 2.

### Comparative Example 2

The same manner as in Example 1 was performed except that β-type zeolite (model number: HSZ940HOD1C, manufactured by Tosoh Corporation) was used instead of ZSM-12. The results are shown in Table 2.

TABLE 1

| | | | Example 1 | Example 2 | Example 3 | Example 4 | Example 5 |
|---|---|---|---|---|---|---|---|
| Catalyst | | | ZSM-12 | ZSM-12 | ZSM-12 | ZSM-12 | ZSM-12 |
| Reaction temperature [°C] | | | 250 | 250 | 250 | 250 | 250 |
| Reaction pressure [MpaG] | | | 0.9 | 0.9 | 0.9 | 0.9 | 0.9 |
| Raw material | Raw material name | | Benzene | Benzene | Benzene | Benzene | Benzene |
| | | | Cyclohexene | Cyclohexene | Cyclohexene | Cyclohexene | Cyclohexanol |
| | Benzene: Cyclohexanol Cyclohexene or (mass ratio) | | 95:5 | 90:10 | 75:25 | 50:50 | 95:5 |
| Reaction product | Conversion [% by mass] | Cyclohexene or Cyclohexanol | 88.0 | 98.4 | 94.8 | 95.0 | 100.0 |
| | Selectivity [% by mass] | cyclohexylbenzene | 74.9 | 53.4 | 43.7 | 33.4 | 58.9 |
| | | Dicyclohexylbenzene | 1.2 | 2.5 | 2.4 | 7.5 | 1.4 |
| | | Dimer other than cyclohexylbenzene | 19.5 | 17.4 | 16.7 | 7.0 | 27.3 |
| | | Bicyclohexyl | 0.0 | 0.0 | 0.0 | 0.2 | 0.0 |
| Cyclohexylbenzene composition | Content [% by mass] | Cyclohexylbenzene | 99.5 | 99.2 | 99.1 | 97.4 | 99.9 |
| | | Bicyclohexyl | 0.0 | 0.1 | 0.1 | 0.6 | 0.0 |

TABLE 2

| | | | Example 6 | Example 7 | Example 8 | Example 9 | Comparative Example 1 | Comparative Example 2 |
|---|---|---|---|---|---|---|---|---|
| Catalyst | | | Silica/alumina | Silica/alumina | ZSM-12 | ZSM-12 | ZSM-5 | β-type zeolite |
| Reaction temperature [°C] | | | 250 | 250 | 250 | 250 | 250 | 250 |
| Reaction pressure [MpaG] | | | 0.9 | 0.9 | 0.9 | 3.0 | 0.9 | 0.9 |
| Raw material | Raw material name | | Benzene | Benzene | Benzene | Benzene | Benzene | Benzene |
| | | | Cyclohexene | Cyclohexanol | Cyclohexene | Cyclohexene | Cyclohexene | Cyclohexene |
| | Benzene: Cyclohexanol Cyclohexene or (mass ratio) | | 95:5 | 95:5 | 95:5 | 95:5 | 95:5 | 95:5 |
| Reaction product | Conversion [% by mass] | Cyclohexene or Cyclohexanol | 99.7 | 100.0 | 93.4 | 99.1 | 29.3 | 11.3 |
| | Selectivity [% by mass] | Cyclohexylbenzene | 24.9 | 64.8 | 60.5 | 70.0 | 24.6 | 17.7 |
| | | Dicyclohexylbenzene | 0.9 | 0.8 | 0.2 | 1.8 | 0.0 | 0.0 |
| | | Dimer other than cyclohexylbenzene | 39.4 | 13.5 | 10.1 | 12.7 | 12.1 | 0.0 |
| | | Bicyclohexyl | 0.0 | 0.0 | 0.0 | 0.0 | 0.0 | 0.0 |
| Cyclohexylbenzene composition | Content [% by mass] | Cyclohexylbenzene | 99.6 | 99.6 | 99.8 | 99.3 | 96.4 | 99.9 |
| | | Bicyclohexyl | 0.0 | 0.0 | 0.0 | 0.0 | 0.0 | 0.0 |

### Discussion

The production methods in Examples 1 to 9 according to the present embodiment were each a method capable of producing cyclohexylbenzene at a high selectivity and a high conversion of cyclohexene or cyclohexanol. On the other hand, the production methods in Comparative Example 1 and Comparative Example 2, in which the MFI-type zeolite catalyst and the β-type zeolite catalyst were respectively used as the solid acid catalysts, exhibited neither a high conversion of cyclohexene or hexanol, nor a high selectivity of cyclohexylbenzene, unlike Examples. It was here confirmed with the production methods in Examples 1 to 4 that a production method in which the content of benzene in the raw material was high tended to allow generation of bicyclohexyl to be more suppressed.

The present application claims the priority based on Japanese Patent Application No. 2020-189699 filed on Nov. 13, 2020, and all the contents described in the Japanese Patent Application are herein incorporated.

1. A method for producing cyclohexylbenzene, comprising a step of bringing a raw material containing benzene and cyclohexene or cyclohexanol into contact with a solid acid catalyst to thereby perform alkylation reaction, wherein the solid acid catalyst is a silica-alumina catalyst or an MTW-type zeolite catalyst.

2. The method for producing cyclohexylbenzene according to claim 1, wherein the solid acid catalyst is a silica-alumina catalyst, and the raw material contains the cyclohexanol.

3. The method for producing cyclohexylbenzene according to claim 1, wherein the solid acid catalyst is an MTW-type zeolite, and the raw material contains the cyclohexene.

4. The method for producing cyclohexylbenzene according to claim 1, wherein the alkylation reaction is performed at a pressure of 0.5 to 3.0 MPaG, a temperature of 150 to 400° C., and a weight hourly space velocity (WHSV) of 1.0 h⁻¹ to 100 h⁻¹.

5. The method for producing cyclohexylbenzene according to claim 1, wherein the raw material contains 70% by mass or more of the benzene.

6. A cyclohexylbenzene composition obtained by the method for producing cyclohexylbenzene according to claim 1, wherein the content of cyclohexylbenzene is 97.0% by mass or more and the content of bicyclohexyl is 1.0% by mass or less.

7. The method for producing cyclohexylbenzene according to claim 2, wherein the alkylation reaction is performed at a pressure of 0.5 to 3.0 MPaG, a temperature of 150 to 400° C., and a weight hourly space velocity (WHSV) of 1.0 h⁻¹ to 100 h⁻¹.

8. The method for producing cyclohexylbenzene according to claim 3, wherein the alkylation reaction is performed at a pressure of 0.5 to 3.0 MPaG, a temperature of 150 to 400° C., and a weight hourly space velocity (WHSV) of 1.0 h⁻¹ to 100 h⁻¹.

9. The method for producing cyclohexylbenzene according to claim 2, wherein the raw material contains 70% by mass or more of the benzene.

10. The method for producing cyclohexylbenzene according to claim 3, wherein the raw material contains 70% by mass or more of the benzene.

11. The method for producing cyclohexylbenzene according to claim 4, wherein the raw material contains 70% by mass or more of the benzene.

12. A cyclohexylbenzene composition obtained by the method for producing cyclohexylbenzene according to claim 2, wherein the content of cyclohexylbenzene is 97.0% by mass or more and the content of bicyclohexyl is 1.0% by mass or less.

13. A cyclohexylbenzene composition obtained by the method for producing cyclohexylbenzene according to claim 3, wherein the content of cyclohexylbenzene is 97.0% by mass or more and the content of bicyclohexyl is 1.0% by mass or less.

14. A cyclohexylbenzene composition obtained by the method for producing cyclohexylbenzene according to claim 4, wherein the content of cyclohexylbenzene is 97.0% by mass or more and the content of bicyclohexyl is 1.0% by mass or less.

15. A cyclohexylbenzene composition obtained by the method for producing cyclohexylbenzene according to claim 7, wherein the content of cyclohexylbenzene is 97.0% by mass or more and the content of bicyclohexyl is 1.0% by mass or less.
